(19) United States
(12) Patent Application Publication
(10) Pub. No.: US 2008/0108697 A1
(43) Pub. Date: May 8, 2008

(54) Device for packaging an oxaliplatin solution

(75) Inventors: Houssam Ibrahim, Veyrier (CH); Ross Blundell, Montferrier Sur Lez (FR); Martin Gray, Northumberland (GB)

Correspondence Address: NIXON & VANDERHYE, PC, 901 NORTH GLEBE ROAD, 11TH FLOOR, ARLINGTON, VA, 22203, US

(73) Assignee: DEBIOPHARM S.A., Lausanne (CH)

(21) Appl. No.: 12/007,010

(22) Filed: Jan. 4, 2008

Related U.S. Application Data
(62) Division of application No. 10/468,915, filed on Aug. 25, 2003.

(30) Foreign Application Priority Data
Mar. 2, 2001 (CH) 389/01

(51) Int. Cl. A61K 31/282; A61P 35/00

(52) U.S. Cl. 514/492

(57) ABSTRACT

The invention concerns an assembly consisting of an aqueous oxaliplatin solution and a glass flask containing same, characterised in that the surface/volume ratio of the flask, expressed in mm²/mm³, is less than 0.26.

The present invention relates to an assembly consisting of an aqueous oxaliplatin solution and a container containing it.

Oxaliplatin (INN; also called I-OHP), a complex derivative of platinum (CAS RN: 61825-94-3) described by Kidani et al. in J. Med. Chem., 1978, 21, 1315, is an antineoplastic agent used intravenously most particularly in the treatment of metastatic colorectal cancers. Currently, it is used in the hospital environment in a lyophilized form and its liquid preparation is reconstituted just before its administration which is carried out generally as an infusion of short duration.

Oxaliplatin, in lyophilized form is formulated with a large quantity of lactose (by a factor of 9 by weight relative to the oxaliplatin). It is then a powder or a cake which is whitish in color. During its reconstitution, it is recommended to use a quantity either of glucose-containing solution, or of a so-called “preparation for injection” (PI) grade water, such that the oxaliplatin concentration in the preparation thus obtained is about 5.0 mg/ml.

Recently, a pharmaceutically stable oxaliplatin preparation, ready to be administered parenterally as an infusion, consisting of an aqueous oxaliplatin solution at a concentration of about 2 mg/ml, and containing no other adjuvants, was described by Ibrahim et al. in WO 96/04904. It is recommended therein to preserve such a liquid preparation in a neutral glass bottle for pharmaceutical use.

This preparation offers hospital staff the great advantage, on the one hand, of no longer having to handle a number of bottles containing either a powder or a cake which is cytotoxic, or the appropriate solvents, during the reconstitution of the pharmaceutical preparation and, on the other hand, of avoiding any risk of using in error a reconstitution solution containing chloride ions, such as a sodium chloride solution normally used in this type of operation, which has the serious consequence of degrading the active substance.

Liquid preparations of oxaliplatin such as those described above can also be preserved in flexible bags for infusion. Mauvernay specified in WO 00/21527 that no degradation was then observed for a period of at least one year, provided that a material free of polyvinyl chloride (PVC) is used as particular plastic material present in direct contact with the liquid preparation of oxaliplatin.

For their part, Anderson et al. observed a tendency of these same aqueous solutions to become degraded over time. To overcome this phenomenon, they proposed in WO 99/43355 to add to these solutions a quantity of a stabilizing agent such as oxalic acid and they recommended preserving the preparations thus obtained in sealed containers such as vials, syringes or flexible bags for infusion. This proposal is however not completely satisfactory because of a degree of toxicity generally attributed to oxalic acid (see The Merck Index, 11th edition, 1989, page 1093).

Health authorities attach a very high importance to pharmaceutical preparations being administered to patients only with a minimum of side effects which could even prove harmful for the health of the patient. Accordingly, they require that it is demonstrated to them through long and fastidious toxicity trials that, when, in a pharmaceutical preparation, the active substance(s) exist(s) in the presence of certain by-products or degradation products, these by-products do not have a deleterious action.

Generally, they tolerate, in a pharmaceutical preparation containing an active substance which has to be administered to a patient in a daily dose of between 100 mg and 2 g, the presence of noncharacterized impurities only if each of these impurities does not exceed a quantity of about 0.2% by weight relative to the weight of the active substance.

As a guide and in the case of a treatment of a patient by administration of oxaliplatin, the dosage generally recommended during a treatment using a short infusion lasting between 2 and 6 hours is between about 85 mg and about 130 mg of oxaliplatin per m² of body surface area. This comes, taking as mean body surface area a value of 1.7 m², to administering daily a dose of between about 145 mg and about 220 mg of oxaliplatin.

Starting with these abovementioned doses to be administered and considering the total number and the respective quantities of degradation products present in a pharmaceutical preparation containing oxaliplatin, the total level of impurities measured should not exceed 2.0% by weight relative to the weight of oxaliplatin after preservation over a period of at least 10 months.

There was therefore a need to find new remedies to these degradations observed over the long term when a preparation of oxaliplatin in solution in water has to be preserved in glass bottles, it being necessary for such remedies, on the one hand, to use only bottles made of materials commonly available on the market and, on the other hand, to exclude the use of chemical stabilizers which may prove to have a deleterious action.

To this effect, the subject of the present invention is the provision of an assembly consisting, on the one hand, of a pharmaceutical preparation of oxaliplatin in aqueous solution and, on the other hand, of a glass bottle containing said preparation, it being necessary for said preparation to satisfy inter alia, in relation to a storage life of at least 10 months, the criteria of purity and/or stability mentioned above.

Said bottle consists of a glass which is normally used for preserving liquid pharmaceutical preparations for parenteral use. It may be obtained according to a so-called “press-and-blow” process, or a so-called “blow-and-blow” process. Preferably, the glass chosen is a so-called type I glass as defined by the American pharmacopeia (United States Pharmacopeia 25—NF 20, 2002) and the European pharmacopeia (Pharmocopée Européenne, 4th edition 2002). Still more preferably, it is a so-called clear or colorless untinted glass. A type II glass, as defined by these same pharmacopeias, can also be used.

This type of glass is particularly recommended for its chemical resistance, in particular its hydrolytic resistance, and its very high chemical durability. It is most particularly suitable for contact with pharmaceutical preparations which are acidic, neutral or alkaline.

This type of glass is based on borosilicate. More specifically, and by way of example, the chemical composition, expressed as a percentage by weight, of some commercial type I glasses is given in table 1 (extract from Technical Methods Bulletin No. 3, Glass containers for small volume parenteral products: Factors for selection and test methods for identification, Parenteral drug association, 1982).

TABLE 1

| | Trade names of the type I glasses | | | | | | |
|---|---|---|---|---|---|---|---|
| | | | | | | | Wheaton |
| Chemical | Kimble | Kimble | Kimble | Wheaton | Wheaton | Wheaton | Type I |
| composition | KG-33 | KG-35 | N51A | NS-33 | NS-51 | NSV | Flint |
| SiO₂ | 80 | 69 | 71 | 81 | 73 | 73 | 70 |
| B₂O₃ | 13 | 13 | 11 | 13 | 10 | 10 | 10 |
| Al₂O₃ | 3 | 6 | 7 | 2 | 6 | 6 | 6 |
| Fe₂O₃ | 0 | 0 | 0 | 0 | 0 | 0 | 0 |
| ZnO | 0 | 0 | 0 | 0 | 0 | 0 | 0.5 |
| TiO₂ | 0 | 0 | 0 | 0 | 0 | 0 | 0 |
| MnO | 0 | 0 | 0 | 0 | 0 | 0 | 0 |
| BaO | 0 | 2 | 2 | 0 | 2 | 2 | 2 |
| CaO | 0 | 1 | 1 | 0 | 1 | 0.5 | 1 |
| MgO | 0 | 0 | 0 | 0 | 0 | 0 | 0.5 |
| Na₂O | 4 | 8 | 6 | 4 | 6 | 7 | 9 |
| K₂O | 0 | 1 | 2 | 0 | 1 | 1 | 1 |

As may be observed, this table suggests that none of the constituents entering into the composition of glass should chemically interfere with the organometallic complex of platinum present in the solution.

In spite of this, the applicant has observed, as did Anderson et al. previously, that substantial degradations sometimes occurred.

In the present case, the oxaliplatin preparations in aqueous solution in which these degradations occurred were nevertheless preserved for a few months at laboratory temperature in glass bottles, in particular in type I glass bottles.

After numerous studies of stability of oxaliplatin preparations in aqueous solution containing no stabilizing agent, as oxalic acid may be for example, and kept under different bottling conditions, the applicant was able to observe, surprisingly, that the stability of these preparations depended on the geometry of the bottles.

More precisely, it was able to show, that on using glass bottles of different shapes, and, for each of the shapes, of different capacities, the existence of a relationship between, on the one hand, the ratio “Surface area of contact of the aqueous oxaliplatin solution with a bottle of a certain capacity/volume for filling said bottle with said oxaliplatin solution” and, on the other hand, the degree of stability of said oxaliplatin solution, a degree of stability characterized by measuring the level of total impurities present in different pharmaceutical preparations contained and preserved in different bottles.

In the remainder of the present application, the term “surface area” will denote the surface area of contact of the aqueous oxaliplatin solution with a glass bottle of a certain capacity and will be expressed in mm², the term “volume” will denote the volume for filling said bottle with said oxaliplatin solution and will be expressed in mm³.

The assembly, according to the present invention, consisting, on the one hand, of a pharmaceutical preparation of oxaliplatin in aqueous solution and, on the other hand, of a glass bottle containing said preparation is characterized in that the surface area/volume ratio is less than 0.26. Preferably, the surface area/volume ratio is less than 0.20.

Moreover, the applicant has been able to determine that the surface area/volume ratio followed the following relationship:
R₀+A.c.Iₘₐₓ
where
R₀ represents the theoretical maximum surface area/volume ratio for which no impurity would be quantifiable (that is to say for Iₘₐₓ=0%) using analytical techniques normally recommended by the pharmacopeia;
A being a constant expressed in ml/(mg.mm);
c representing the oxaliplatin concentration expressed in mg/ml; and
Iₘₐₓ representing the noncharacterized maximum level of total impurities by weight accepted.

The invention will be described more precisely with the aid of the following examples and of the drawing in which:

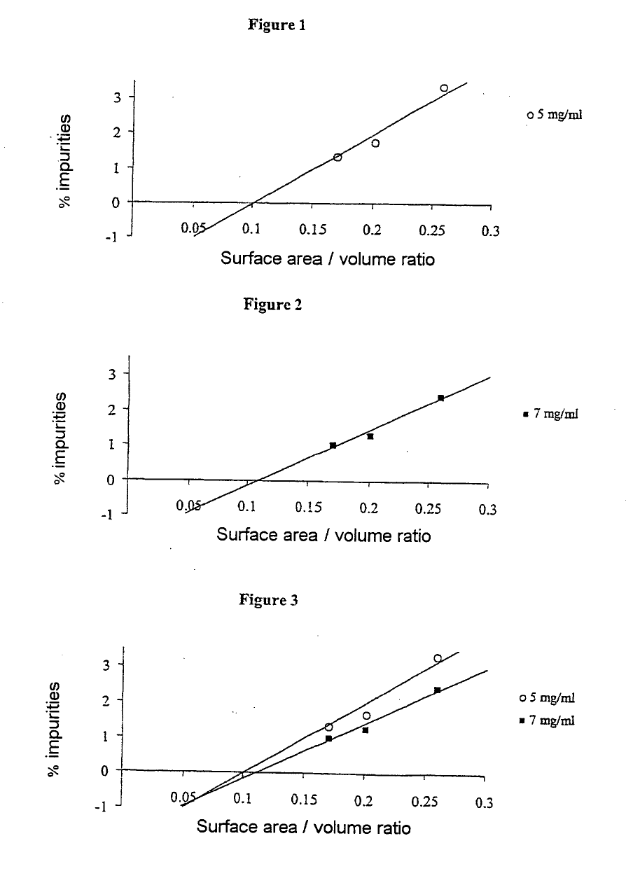

FIG. 1 represents the level of noncharacterized total impurities by weight in an aqueous preparation of oxaliplatin at a concentration of 5 mg/ml after 4 months of storage as a function of the surface area/volume ratio;

FIG. 2 represents the level of noncharacterized total impurities by weight in an aqueous preparation of oxaliplatin at a concentration of 7 mg/ml after 4 months of storage as a function of the surface area/volume ratio;

FIG. 3 shows a superposition of the curves illustrated in FIGS. 1 and 2;

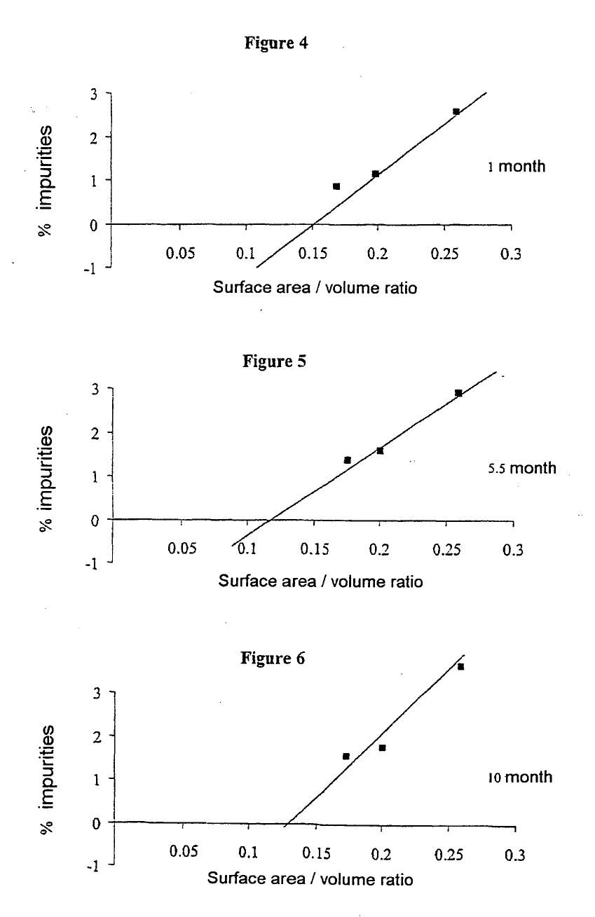

FIG. 4 represents the level of noncharacterized total impurities by weight in an aqueous preparation of oxaliplatin at a concentration of 5 mg/ml after 1 month of storage as a function of the surface area/volume ratio;

FIG. 5 represents the level of noncharacterized total impurities by weight in an aqueous preparation of oxaliplatin at a concentration of 5 mg/ml after 5.5 months of storage as a function of the surface area/volume ratio;

FIG. 6 represents the level of noncharacterized total impurities by weight in an aqueous preparation of oxaliplatin at a concentration of 5 mg/ml after 10 months of storage as a function of the surface area/volume ratio;

### 1: PREPARATION AND STORAGE OF THE SAMPLES

To carry out this trial, four series of bottles, consisting of a colorless type I glass, all of cylindrical shape but of different volumes, were used. Table 2 assembles, for each series of bottles, their so-called “useful” capacity, their so-called “brim” capacity, the inner diameter of these bottles, that of their neck and their height.

TABLE 2

| | Useful | Brim | Inner | Neck | |
|---|---|---|---|---|---|
| | capacity | capacity | diameter | diameter | Height |
| Series | (ml) | (ml) | (mm) | (mm) | (mm) |
| 1 | 5 | 7 | 23.50 | 20.0 | 40.0 |
| 2 | 15 | 17 | 29.90 | 20.0 | 60.0 |
| 3 | 20 | 22 | 29.90 | 20.0 | 60.0 |
| 4 | 50 | 60 | 42.47 | 20.0 | 70.0 |

These bottles, used for the first time, were subjected beforehand to three cycles of washing and rinsing with hot water heated to about 50° C. and water of so-called PI grade before being dried.

Three oxaliplatin stock solutions at concentrations of 2 mg/ml, 5 mg/ml and 7 mg/ml, respectively, were prepared in the usual manner using PI grade water as solvent. No particular stabilizing agent was used.

Aliquots of these preparations were collected and then transferred under aseptic filling conditions into different bottles so as to reach the respective level corresponding to the heights indicated below. The bottles were then hermetically closed by seaming a cap.

For studies of stability under normal conditions, a first portion of these bottles were then placed in a first chamber thermostated at a temperature of 25° C. and at a relative humidity of 60%. These bottles were maintained upright and at rest without particular stirring for the periods indicated below.

Samples were collected at the periods indicated and then analyzed by high-performance liquid chromatography according to a conventional method in order to quantify the level of noncharacterized total impurities, expressed as a percentage by weight, relative to the quantities of oxaliplatin present in each of the samples.

### 2. RESULTS OF THE STUDY OF THE STABILITY OF PREPARATIONS OF OXALIPLATIN IN AQUEOUS SOLUTION AT A CONCENTRATION OF 5 MG/ML

This study was performed on groups of bottles having a useful capacity of 5 ml, 15 ml and 20 ml, respectively, filled as described above with aliquots of a stock solution at a concentration of 5 mg/ml, and then stored under the conditions mentioned above for a period of at least 10 months.

Samples were collected at periods of 1 month, and then 2.5 months, 4 months, 5.5 months, 7 months and 10 months, respectively, after bottling.

Table 3 below assembles, for each of the bottles having a respective useful capacity of 5 ml, 10 ml and 15 ml, the inner diameter of the bottle, the height for filling with the liquid preparation, the volume for filling with aqueous preparation and the calculated surface area of the walls of the bottle in contact with this aqueous preparation and then the surface area/volume ratio. Table 4 assembles, for each of the bottles, the level of total impurities measured at a given time indicated and expressed as a percentage by weight relative to the quantity of oxaliplatin present.

TABLE 3

| | | | | Surface | Surface |
|---|---|---|---|---|---|
| Useful | Inner | Filling | Filling volume | area of | area/ |
| capacity | diameter | height | ×10³ ± 4% | contact | volume |
| (ml) | (mm) | (mm) | (mm³) | ×10² (mm²) | ratio |
| 5 | 23.50 | 10.58 | 4.59 | 12.15 | 0.26 |
| 15 | 29.90 | 15.55 | 10.92 | 21.63 | 0.20 |
| 20 | 29.90 | 30.51 | 21.42 | 35.67 | 0.17 |
| 50 | 42.47 | 35.30 | 50.00 | 61.25 | 0.12 |

TABLE 4

| | Level | | | | | |
|---|---|---|---|---|---|---|
| | of impurity | Level | | Level of | | Level of |
| | (% by | of impurity | Level | impurity | Level of | impurity |
| Useful | weight) | 2.5 | of impurity | 5.5 | impurity | 10 |
| capacity | 1 month | months | 4 months | months | 7 months | months |
| 5 ml | 2.34 | 2.55 | 2.89 | 2.70 | 3.19 | 3.64 |
| 15 ml | 1.15 | 1.16 | 1.23 | 1.50 | 1.56 | 1.59 |
| 20 ml | 1.06 | 1.11 | 1.13 | 1.38 | 1.42 | 1.45 |

It is noted that the bottle having a useful capacity of 5 ml is not satisfactory because the noncharacterized maximum level of total impurities accepted, that is 2.0%, is already exceeded when the initial analysis carried out 1 month after dissolving in solution.

FIG. 1 represents the values of the “4 months” column of Table 4 as a function of the surface area/volume ratio.

### 3. RESULTS OF THE STUDY OF THE STABILITY OF PREPARATIONS OF OXALIPLATIN IN AQUEOUS SOLUTION AT A CONCENTRATION OF 7 MG/ML

This study was performed as above, with the difference that the bottles were loaded with aliquots of a stock solution at a concentration of 7 mg/ml and samples were collected at the same periods.

Table 5 below assembles, for each of the bottles, the level of total impurities measured at a given moment indicated and expressed as a percentage by weight relative to the quantity of oxaliplatin present.

TABLE 5

| | Level | | | | | |
|---|---|---|---|---|---|---|
| | of impurity | Level | | Level of | | Level of |
| | (% by | of impurity | Level | impurity | Level of | impurity |
| Useful | weight) | 2.5 | of impurity | 5.5 | impurity | 10 |
| capacity | 1 month | months | 4 months | months | 7 months | months |
| 5 ml | 1.87 | 2.09 | 2.33 | 2.56 | 2.75 | 2.98 |
| 15 ml | 0.96 | 1.03 | 1.12 | 1.19 | 1.23 | 1.30 |
| 20 ml | 0.70 | 0.81 | 0.97 | 1.04 | 1.07 | 1.11 |

As in the trial carried out on the aqueous preparation of oxaliplatin at a concentration of 5 mg/ml, it is also noted that the bottle having a useful capacity of 5 ml is not satisfactory. However, the maximum level of noncharacterized total impurities accepted is only exceeded later. The result is that the stability of the solution increases with the concentration.

FIG. 2 represents the values of the “4 months” column of table 5 as a function of the surface area/volume ratio.

FIG. 3 represents a superposition of the curves of FIGS. 1 and 2, which makes it possible to better illustrate the fact that the stability of the solution increases with the concentration.

FIGS. 4 to 6 represent the values of the “1 month”, “5.5 months” and “10 months” columns of table 5 as a function of the surface area/volume ratio.

### 4. RESULTS OF THE STUDY OF LONG-TERM STABILITY OF PREPARATIONS OF OXALIPLATIN AQUEOUS SOLUTION AT A CONCENTRATION OF 2 MG/ML

This study was performed on three batches of bottles having the same useful capacity of 50 ml, filled as described above with aliquots of the same volume of a stock solution at a concentration of 2 mg/ml, and then stored under the conditions mentioned above for a period of 5 years. At the end of this period, samples were collected for analysis.

Table 6 below assembles, for these bottles of the same useful capacity, their inner diameter, the height for filling with the liquid preparation, the volume for filling with aqueous preparation and the calculated surface area of the walls of the bottle in contact with this aqueous preparation and then the surface area/volume ratio. Table 7 assembles, for each of the bottles, the level of total impurities measured after 5 years.

TABLE 6

| | | | | Surface | Surface |
|---|---|---|---|---|---|
| Useful | Inner | Filling | Filling volume | area of | area/ |
| capacity | diameter | height | ×10³ ± 4% | contact | volume |
| (ml) | (mm) | (mm) | (mm³) | ×10² (mm²) | ratio |
| 50 | 42.47 | 35.30 | 50.00 | 61.25 | 0.12 |

TABLE 7

| | Level of impurity (% by weight) |
|---|---|
| Batch | 5 years |
| 1 | 1.47 |
| 2 | 1.56 |
| 3 | 1.55 |

### 5. COMMENTS AND CONCLUSIONS

From the teaching of FIGS. 1 and 2, it is noted that the level of noncharacterized total impurities decreases when the surface area/volume ratio decreases.

Even from the beginning of the storage of the bottles of oxaliplatin, the stability of the solution is better for a low surface area/volume ratio.

In addition, a linear relationship is observed between the surface area/volume ratio and the level of impurities.

Taking into account the results presented above, the following general equation may be deduced:
R=R₀+A.c.I
where
I represents the level of noncharacterized total impurities present in the aqueous preparation of oxaliplatin at a given concentration;
R₀ represents the theoretical maximum surface area/volume ratio for which no impurity would be quantifiable (that is to say for Iₘₐₓ=0%) using analytical techniques normally recommended by the pharmacopeia, this value being dependent on the oxaliplatin concentration in the preparation;
A is a constant expressed in ml/(mg.mm);
c represents the oxaliplatin concentration in the preparation expressed in mg/ml; and
R represents the surface area/volume ratio specific to the bottle considered at a given filling.

Taking the results illustrated in FIG. 1, the following values can be deduced from the curve:
A=0.01 ml/(mg.m) and R₀=0.10 for c=5 mg/ml

Taking the results illustrated in FIG. 2, the following values can be deduced from the curve:
A=0.009 ml/(mg.m) and R₀=0.11 for c=5 mg/ml

It should be noted moreover, as can be observed in FIGS. 4 to 6, that the stability of the preparation decreases linearly over time.

It thus becomes possible to choose an appropriate surface area/volume ratio, for example 0.1, when a given storage time is set, for example 3 years.

In practice, to determine the surface area/volume ratio not to be exceeded for a given bottle containing a pharmaceutical preparation of oxaliplatin as an aqueous solution at a given concentration, the procedure may be carried out in the following manner:

At least two bottles of similar shape but of different volumes (therefore of different surface area/volume ratios) are used, and they are filled with the aqueous preparation of oxaliplatin.

The surface area/volume ratios are then determined, and then the respective levels of noncharacterized total impurities are quantified at given periods of storage (for example at 1 month or 4 months). A graph is then established on which the levels of impurities measured are plotted as a function of the “surface area/volume” ratio and the place where the x-axis and the curve cross is determined. The value thus obtained gives the surface area/volume ratio which should not be exceeded.

The applicant has further observed that this invention is particularly effective for a filling volume greater than 7 ml. Preferably, the present invention is applicable to any oxaliplatin solution contained in a bottle having a useful capacity equal to or greater than 10 ml.

Preferably, the pharmaceutical preparations of oxaliplatin stored are those in which the oxaliplatin is in aqueous solution at concentrations of between 2 and 7 mg/ml.

Finally, it will be noted that the applicant has carried out a study of stability under accelerated conditions intended to anticipate stabilities on a scale of 3 years. To this effect, bottles were placed in a chamber thermostated at a temperature of 40° C. and in an atmosphere having a humidity of 75%. Samples were regularly collected and then analyzed.

The results obtained suggest that pharmaceutical preparations of oxaliplatin in aqueous solution may be stored in the bottles already selected above and under the filling conditions indicated for a period ranging up to at least 36 months, thus meeting the best periods of storage recognized by health authorities.

However, it goes without saying that the specialist will know how to apply the invention without being limited either to the concentrations used, or to the shapes of the bottles (bottles with a parellelepipedal or cylindrical base) or to the types of glass used in the preceding examples. In addition, the invention is applicable to any pharmaceutical preparation of oxaliplatin in aqueous solution, it being possible for the latter to further contain components such as stabilizing agents (e.g. buffering agents).

1-6. (canceled)

7. A method of storing a pharmaceutical preparation of oxaliplatin in aqueous solution comprising:
using a glass bottle containing the pharmaceutical preparation of oxaliplatin in aqueous solution at a concentration between 2 mg/ml and 7 mg/ml, wherein the bottle has a surface area/volume ratio, expressed in mm²/mm³, of less than 0.20 and which useful capacity is equal to or greater than 10 ml;
maintaining during at least 10 months said preparation with a total level of impurity not exceeding 2% by weight relative to the weight of the oxaliplatin,
said surface area being the surface area of contact of the aqueous oxaliplatin solution with the bottle, and said volume being the volume for filling the bottle with said oxaliplatin solution.

8. The method of claim 7, wherein the concentration of the pharmaceutical preparation of oxaliplatin in aqueous solution is 2 mg/ml.

9. The method of claim 7, wherein the concentration of the pharmaceutical preparation of oxaliplatin in aqueous solution is 5 mg/ml.

10. The method of claim 7, wherein said bottle is made of Type I glass.
